(19) United States
(12) Patent Application Publication
(10) Pub. No.: US 2026/0315238 A1
(43) Pub. Date: Oct. 15, 2026

(54) ACETABULAR PROSTHETIC DEVICE

(71) Applicant: LIMACORPORATE S.P.A., San Daniele Del Friuli (UD) (IT)

(72) Inventors: Federico LEANDRIN, CORDOVADO (PN) (IT); Michele PRESSACCO, MARTIGNACCO (UD) (IT); Thomas FERRO, MORTEGLIANO (UD) (IT)

(21) Appl. No.: 19/483,475

(22) PCT Filed: May 24, 2024
(86) PCT No.: PCT/IT2024/050105

§ 371 Date: Nov. 11, 2025

(30) Foreign Application Priority Data
May 25, 2023 (IT) 102023000010662
Sep. 5, 2023 (IT) 102023000018252

(51) Int. Cl. A61F 2/34; A61F 2/30

(52) U.S. Cl. CPC A61F 2/34; A61F 2/30744; A61F 2/30749; A61F 2002/30245; A61F 2002/30332; A61F 2002/30378; A61F 2002/30405

(57) ABSTRACT

Acetabular prosthetic device (10) comprising an acetabular cup (12) provided with a through aperture configured to receive both an iliac stem (11) and also a safety cap (13).

### FIELD OF THE INVENTION

The present invention concerns an acetabular prosthetic device used to restore the correct functioning of the hip joint, for example in the event a patient's pelvis presents notable bone deformities or cavities of a natural origin, for example due to osteoarthritis, tumor, dysplasia, and/or of an artificial origin, for example caused by the removal of a previous prosthetic implant.

### BACKGROUND OF THE INVENTION

Acetabular prosthetic devices are generally formed by an acetabular cup configured to be stably attached in a patient's acetabulum. To guarantee a correct attachment, the acetabular cup can be associated with a stem that is inserted into the iliac canal of the pelvis. In some cases, this stem can be attached to the patient's sacrum, as a replacement for or in addition to the anchoring in the iliac canal. Regardless of where the anchoring takes place, this type of stem is, here and hereafter, called “iliac stem”.

There are known acetabular prosthetic devices with an iliac stem made in a single body with the acetabular cup, or with an iliac stem that can be coupled to the external surface of the acetabular cup by means of threaded attachment means, before inserting the cup and stem into the patient's acetabular cavity.

A disadvantage of prosthetic devices that have the acetabular cup with integrated or pre-assembled stem is that they have significant mass and overall dimensions that complicate the surgical operation.

Another disadvantage of the aforementioned prosthetic devices with stem either integrated or pre-mounted on the acetabular cup is that the position of the acetabular cup, during surgery, could be different from the one originally planned by the surgeon, and the iliac stem, whether integrated or rigidly clamped onto the acetabular cup, is therefore also incorrectly positioned, that is, in a bone portion with low bone density.

Acetabular prosthetic devices are also known in which the iliac stem can be inserted from the inside, that is, from the concave side, of the acetabular cup after the insertion of the acetabular cup into the patient's acetabular cavity.

A disadvantage of these acetabular prosthetic devices is that the iliac stem cannot be screwed to the acetabular cup because a rotation of the stem would induce an axial torsion of the tissues present in the iliac canal, with the risk of losing contact between the prosthetic implant and the anatomy.

Another disadvantage of these acetabular prosthetic devices is that the screwing of the iliac stem into the iliac canal can result in fractures in the pelvis.

Therefore, currently the iliac stems that can be inserted from the inside of the cup are attached to the acetabular cup only by means of a flat head abutment and secured through compression by a safety or locking screw. These stems, however, do not guarantee a stable attachment since the displacement of the stem along its axis is disadvantageously unconstrained. Stems of this type, therefore, can mobilize during use and cause pain and serious injuries to the patient.

Other known solutions for stably attaching the acetabular cup in the patient's acetabulum provide, as an alternative or in addition to the iliac stem, the use of locking screws that have a first threading profile able to attach inside the anatomy, compressing the patient's spongy bone, and a second threading profile able to attach on a threaded seating of the acetabular cup.

The locking screws are inserted into the patient's anatomy downstream of the acetabular cup positioning. This allows to first insert the acetabular cup in the correct position, and then insert the locking screws, resulting in a “pack-closure” of the acetabular cup with the acetabulum.

Disadvantageously, this solution does not allow to adjust the position of the axis of the locking screws with respect to the axis of the corresponding holes present in the acetabular cup, and neither to obtain an adequate coupling to the acetabulum and/or to correctly trigger osseointegration phenomena.

Document WO 2010/072604 A1 describes a stabilization element for endoprostheses able to be polyaxially aligned.

Document WO 2009/081346 A1 describes an acetabular prosthesis comprising a cup able to be inserted into a corresponding acetabulum and a cup attachment screw able to engage by screwing into a portion of pelvic bone.

Document DE 19542116 A1 describes a device for attaching an implant to a bone.

Document U.S. Pat. No. 5,888,204 A describes an acetabular cup having a polyaxial locking screw for anchoring the cup in the desired bone site.

There is therefore the need to perfect an acetabular prosthetic device that can overcome at least one of the disadvantages of the state of the art.

One purpose of the present invention, which corresponds to the technical problem that the Applicant intends to solve, is to provide an acetabular prosthetic device, of the type with an iliac stem able to be inserted from inside the acetabular cup, in which the coupling between the iliac stem and the acetabular cup prevents axial displacements of the stem and is obtained without applying torsion/rotation on the stem itself.

In particular, one purpose of the present invention is to prevent axial displacements of the stem with respect to the acetabular cup following the determination of the reciprocal axial coupling by the surgeon.

Another purpose of the present invention is to provide an acetabular prosthetic device in which the axis of the stem can be oriented angularly with respect to the acetabular cup before the reciprocal axial coupling is determined.

The Applicant has devised, tested and embodied the present invention to overcome the shortcomings of the state of the art and to obtain these and other purposes and advantages.

### SUMMARY OF THE INVENTION

The present invention is set forth and characterized in the independent claims. The dependent claims describe other characteristics of the present invention or variants to the main inventive idea.

In accordance with the above purposes and to resolve the technical problem disclosed above in a new and original way, also achieving considerable advantages compared to the state of the prior art, an acetabular prosthetic device according to the present invention comprises an acetabular cup provided with a through aperture configured to receive both an iliac stem and also a safety element, that is, a safety cap. The iliac stem, the acetabular cup and the safety cap form the acetabular prosthetic device.

In accordance with one aspect of the present invention, the iliac stem has a first end provided both with a first coupling portion conformed to be accommodated in a mating first coupling seating defined in the aperture, and also with an abutment surface against which the safety cap can abut, the safety cap being conformed to be accommodated in a mating second coupling seating defined in the aperture.

Doing so achieves at least the advantage of being able to insert the iliac stem from inside the acetabular cup, downstream of the correct positioning of the acetabular cup in the patient's acetabular cavity. Furthermore, thanks to the presence of the safety cap, which pack-closes the iliac stem in the first coupling seating through pressure on the stem's abutment surface, it is possible to avoid any torsional stress on the stem.

According to a variant, the first coupling seating and the first coupling portion can have a mating truncated conical shape. The conical coupling between the first coupling portion and the first coupling seating allows for a univocal axial positioning between iliac stem and acetabular cup.

In accordance with another aspect of this variant, the abutment surface of the iliac stem is at least partly flat, and the safety cap can abut against it with an essentially flat contact surface thereof. This configuration of contact between safety cap and iliac stem allows for an orientation of the iliac stem along a single axis regardless of the spherical or conical coupling between stem and acetabular cup.

In accordance with another aspect of the present invention, the first coupling seating and the first coupling portion can have a mating at least partly spherical shape. The spherical coupling between the first coupling portion and the coupling seating can allow for a “polyaxial” positioning, that is, along a multiplicity of axes, between iliac stem and acetabular cup, before the stem is clamped with respect to the acetabular cup by means of the safety cap.

This aspect of the invention advantageously allows the iliac stem to be inserted inside the iliac canal with an optimal positioning, that is, in a zone with greater bone density, even in the event the acetabular cup is firmly attached but in a different position from what was originally planned by the surgeon.

In accordance with another aspect of the present invention, the first end of the iliac stem has a concave receiving portion configured to receive a mating central protrusion which protrudes from the contact surface of the safety cap.

In accordance with another aspect of the present invention, the receiving portion can have a truncated conical shape and the central protrusion can be at least partly spherical or truncated conical.

According to a variant, the receiving portion can have an at least partly spherical shape and the central protrusion can have a corresponding at least partly spherical shape.

Advantageously, when the safety cap has the central protrusion, the “polyaxial” positioning between iliac stem and acetabular cup is permitted only if the receiving portion has a truncated conical shape and the central protrusion has an at least partly spherical shape, or if the receiving portion and the central protrusion both have an at least partly spherical shape, given the spherical coupling between iliac stem and acetabular cup.

In accordance with another aspect of the present invention, the second coupling seating is provided with a threaded segment and the safety cap is provided with a corresponding peripheral threaded segment, in order to determine a threaded coupling between the two components. It is therefore clear that the torsional action only involves the acetabular cup and the safety cap, while the pressure action on the iliac stem occurs only in the axial direction.

Some embodiments of the present invention also concern an iliac stem for an acetabular prosthetic device having a first end provided both with a first coupling portion, with a truncated conical or at least partly spherical shape, and also with a frontal abutment surface from which a concave receiving portion develops internally having a truncated conical or at least partly spherical shape.

### DESCRIPTION OF THE DRAWINGS

These and other aspects, characteristics and advantages of the present invention will become apparent from the following description of some embodiments, given as a non-restrictive example with reference to the attached drawings wherein:

FIG. 1 is a schematic view that shows the use of an acetabular prosthetic device, according to the present invention;

FIGS. 2-6 are enlarged partial section views of some embodiments of the acetabular prosthetic device of FIG. 1.

We must clarify that the phraseology and terminology used in the present description, as well as the figures in the attached drawings also in relation as to how described, have the sole function of better illustrating and explaining the present invention, their purpose being to provide a non-limiting example of the invention itself, since the scope of protection is defined by the claims.

To facilitate comprehension, the same reference numbers have been used, where possible, to identify identical common elements in the drawings. It is understood that elements and characteristics of one embodiment can be conveniently combined or incorporated into other embodiments without further clarifications.

### DESCRIPTION OF SOME EMBODIMENTS OF THE PRESENT INVENTION

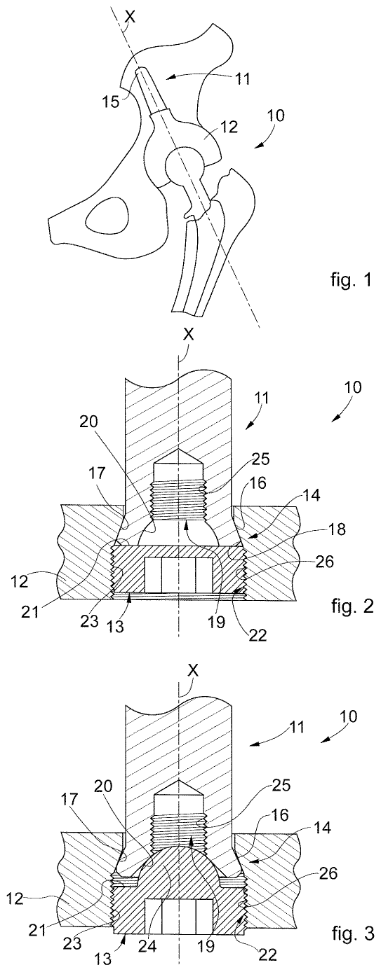

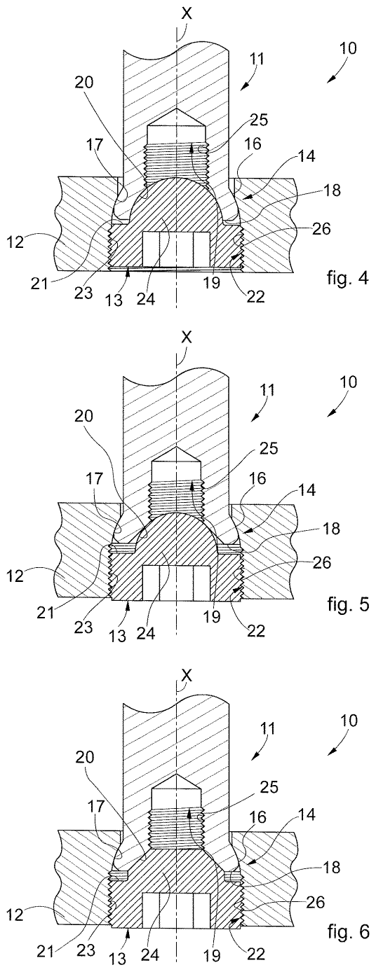

With reference to FIG. 1, an acetabular prosthetic device 10 comprises an acetabular cup 12, stably attached in a patient's acetabulum, and an iliac stem 11 inserted into the iliac canal of the patient's pelvis. The device 10 also comprises a safety cap 13, visible in FIGS. 2-6, configured to determine an axial coupling between the iliac stem 11 and the acetabular cup 12.

By way of example, the iliac stem 11 has a cylindrical shape. However, a person of skill in the art will understand that the shape of the iliac stem 11 is not limited to the cylindrical one and may have one or more conical segments, and/or one or more attachment tabs on a lateral surface thereof.

The iliac stem 11 can be made of metallic material, preferably titanium or an alloy thereof.

According to possible embodiments, the iliac stem 11 can be coated completely or in part with a layer of titanium pore and/or hydroxyapatite in order to promote osseointegration with the bone.

The iliac stem 11 develops along a longitudinal axis X and has a first end, or head, 14 and an opposed second end, or tip, 15, the latter visible in FIG. 1.

The first end 14 is conformed to couple to the acetabular cup 12 and to cooperate with the safety cap 13 while the second end 15 is the “first insertion” portion of the iliac stem 11 into the patient's iliac canal.

The first end 14 has a first coupling portion 16 conformed to couple to a mating first coupling seating 17 present in the acetabular cup 12.

According to some embodiments, the first coupling portion 16 has a conical/truncated conical shape, FIGS. 2 and 3. Correspondingly, the first coupling seating 17 also has a mating conical/truncated conical shape.

The conical coupling between the first coupling portion 16 and the first coupling seating 17 allows for a univocal axial positioning between iliac stem 11 and acetabular cup 12. In other words, the longitudinal axis X of the iliac stem 11 coincides with the axes around which the first coupling portion 16 and the first coupling seating 17 are defined.

The taper of the first coupling portion 16 and of the first coupling seating 17 can be slightly different so as to improve the axial coupling by interference (FIG. 3).

According to other embodiments shown in FIGS. 4-6, the first coupling portion 16 has an at least partly spherical shape. Correspondingly, the first coupling seating 17 also has a mating at least partly spherical shape.

In the examples of FIGS. 4 and 5, the first end 14 of the iliac stem 11 is configured to articulate in an angularly orientable manner in the first coupling seating 17.

The spherical coupling between the first coupling portion 16 and the first coupling seating 17 allows for a “polyaxial” positioning, that is, along a multiplicity of axes, between iliac stem 11 and acetabular cup 12. In this case, in fact, an inclination, or orientation, of the iliac stem 11 in the first coupling seating 17 is allowed before the position is blocked by means of the safety cap 13, as will be described below.

According to some embodiments, the first end 14 of the iliac stem 11 has a frontal abutment surface 18 against which the safety cap 13 can abut, FIG. 2.

According to some embodiments, the abutment surface 18 can be at least partly flat. In this case, a polyaxial adjustment is not allowed since the contact of the safety cap 13 against the abutment surface 18 determines a univocal positioning, regardless of the conical or spherical coupling between iliac stem 11 and acetabular cup 12.

Advantageously, a cavity 19 is made in the body of the iliac stem 11 which develops along the longitudinal axis X toward the second end 15. The cavity 19 can be provided with a threaded segment 25. The cavity 19 is configured to receive a surgical impactor, guided to move and position the iliac stem 11 during surgery.

According to some embodiments, the first end 14 of the iliac stem 11 can have a hollow receiving portion 20, having a concave conformation that develops inside the body of the iliac stem 11.

The receiving portion 20 develops around the longitudinal axis X starting from the abutment surface 18. The receiving portion 20 substantially forms an extension of the abutment surface 18.

The receiving portion 20 is located upstream with respect to the cavity 19, in an entrance zone of the first end 14.

The receiving portion 20 is configured to cooperate with the safety cap 13, in particular to receive a central protrusion 24 of the safety cap 13, as will be described in more detail below.

According to some embodiments, the receiving portion 20 can have a spherical or hemispherical shape (FIGS. 2-5), or it can have a truncated conical shape, coaxial with the body of the iliac stem 11 (FIG. 6).

The iliac stem 11 is constrained within the first coupling seating 17 by the safety cap 13, which is configured to prevent the axial displacement of the iliac stem 11 with respect to the acetabular cup 12.

The safety cap 13 is configured to be inserted in a second coupling seating 22 of the acetabular cup 12 to translate in an axial direction toward the first end 14 of the iliac stem 11.

The second coupling seating 22 has, by way of example, a cylindrical shape mating with the shape of the safety cap 13.

The first coupling seating 17 together with the second coupling seating 22 defines an aperture, passing through the hemispherical wall that defines the acetabular cup 12. Advantageously, the first coupling seating 17 and the second coupling seating 22 can be completely created in the thickness of said hemispherical wall.

According to one embodiment, the second coupling seating 22 of the acetabular cup 12 can have a threaded segment 26, and the safety cap 13 can be provided with a corresponding peripheral threaded segment 23 to allow, by screwing, a translation of the safety cap 13 inside the second coupling seating 22 in an axial direction toward the iliac stem 11.

According to some embodiments, the second coupling seating 22 can advantageously have a “female” thread of the self-locking type.

The safety cap 13 is provided with an at least partly flat contact surface 21.

The contact surface 21 faces, during use, toward the abutment surface 18 or toward the receiving portion 20 in order to “pack-tighten” the iliac stem 11 against the first coupling seating 17 and constrain the iliac stem 11 in position, preventing an axial displacement thereof.

According to some embodiments, the contact surface 21 is completely flat (FIG. 2).

According to other embodiments, the contact surface 21 can advantageously have a central protrusion 24 which, during use, is able to come into contact with the receiving portion 20 of the iliac stem 11, FIGS. 3-6. A part of the flat or inclined contact surface, provided annularly around the central protrusion 24, remains distanced from the abutment surface 18 of the iliac stem 11 so as to create a gap, or clearance, to allow the angular orientation of the iliac stem 11.

The central protrusion 24 can have a hemispherical shape. The receiving portion 20 can, in this case, have an at least partly spherical shape, FIGS. 2 and 4. Alternatively, the receiving portion 20 can have a truncated conical shape (FIGS. 3 and 5). This coupling configuration between safety cap 13 and iliac stem 11 allows the iliac stem 11 to be oriented angularly (polyaxiality).

According to an embodiment described in FIG. 5, the first end 14 of the iliac stem 11 is at least partly spherical, mating with the at least partly spherical first coupling portion 16. The iliac stem 11 can advantageously be oriented angularly with respect to the axis that defines the first coupling portion 16 and the first coupling seating 17 to provide the surgeon, during use, with the most correct positioning for the iliac stem 11.

The receiving portion 20 of the embodiment described in FIG. 5, moreover, is truncated conical, while the safety cap 13 has the central protrusion 24. This combination advantageously allows to pack-tighten the safety cap 13 in the second coupling seating 22 against the iliac stem 11, whatever the position of the iliac stem 11 with respect to the axis that defines the first coupling portion 16 and the first coupling seating 17. The at least partial sphericity of the central protrusion 24 allows to maximize the contact surface against the truncated conical receiving portion 20, thus increasing the tightness of the safety cap 13.

According to a variant, the central protrusion 24 can have a truncated conical shape, FIG. 6. The receiving portion 20 can, in this case, have a mating truncated conical shape. This coupling configuration between safety cap 13 and iliac stem 11 does not allow the iliac stem 11 to be oriented angularly.

By screwing the safety cap 13, the central protrusion 24 pack-tightens the iliac stem 11 against the first coupling seating 17 of the acetabular cup 12 and constrains it in position, preventing any axial displacements.

The polyaxiality between iliac stem 11 and acetabular cup 12 advantageously allows to offer the surgeon wide versatility during surgery. For example, in the event that the surgeon is able to securely attach the acetabular cup 12 in the planned position and orientation, the surgeon can insert the iliac stem 11 into the first coupling seating 17 of the acetabular cup 12 so that the longitudinal axis X of the iliac stem 11 is coincident with the axis that defines the first coupling portion 16 and the first coupling seating 17.

The surgeon can clamp the iliac stem 11 along the same axis by means of an embodiment of the safety cap 13 in accordance with the present invention that determines a univocal positioning, regardless of the spherical coupling between iliac stem 11 and acetabular cup 12, for example the safety cap 13 having a truncated conical protrusion shown, by way of a non-limiting example, in FIG. 6.

Instead, in the event that the surgeon securely attaches the acetabular cup 12 in a position and/or orientation different from the one originally planned, the surgeon can vary the angular positioning of the iliac stem 11 with respect to the first coupling seating 17 of the acetabular cup 12, and clamp the iliac stem 11 in the angular position identified by means of an embodiment of the safety cap 13 in accordance with the present invention that allows polyaxiality, for example, the safety cap 13 having an at least partly spherical protrusion, as in the example shown, purely by way of a non-limiting example, in FIG. 5.

It is clear that modifications and/or additions of parts may be made to the acetabular prosthetic device 10 as described heretofore, without thereby departing from the field and scope of the present invention, as defined by the claims.

It is also clear that, although the present invention has been described with reference to some specific examples, a person of skill in the art will be able to achieve other equivalent forms of acetabular prosthetic device, having the characteristics as set forth in the claims and hence all coming within the field of protection defined thereby.

In the following claims, the sole purpose of the references in brackets is to facilitate their reading and they must not be considered as restrictive factors with regard to the field of protection defined by the claims.

1. An acetabular prosthetic device (10) comprising:
an acetabular cup (12) provided with a through aperture configured to receive both an iliac stem (11) and also a safety cap (13),
wherein said iliac stem (11) has a first end (14) provided both with a first coupling portion (16) conformed to be accommodated in a mating first coupling seating (17) defined in said aperture, and also with an abutment surface (18) against which said safety cap (13) can abut, said safety cap (13) being conformed to be accommodated in a mating second coupling seating (22) defined in said aperture, wherein said first coupling seating (17) and said first coupling portion (16) have a mating truncated conical shape.

2. The device (10) as in claim 1, wherein said first coupling seating (17) and said first coupling portion (16) are configured to define a reciprocal conical coupling which defines a univocal axial positioning between iliac stem (11) and acetabular cup (12).

3. The device (10) as in claim 1, wherein said first end (14) of said iliac stem (11) has a concave receiving portion (20) configured to receive a central protrusion (24) which protrudes from a contact surface (21) of said safety cap (13).

4. The device (10) as in claim 3, wherein said receiving portion (20) has a truncated conical shape.

5. The device (10) as in claim 3, wherein said receiving portion (20) has an at least partly spherical shape.

6. The device (10) as in claim 3, wherein said central protrusion (24) has a truncated conical shape.

7. The device (10) as in claim 3, wherein said central protrusion (24) has an at least partly spherical shape.

8. The device (10) as in claim 1, wherein said second coupling seating (22) is provided with a threaded segment (26) and said safety cap (13) is provided with a corresponding peripheral threaded segment (23), in order to determine a threaded coupling.

9. An iliac stem (11) for an acetabular prosthetic device (10) as in claim 1, wherein it has a first end (14) provided both with a first coupling portion (16) with a truncated conical shape, and also with a frontal abutment surface (18) from which a concave receiving portion (20) develops internally having a mating truncated conical shape.
